(19) United States
(12) Patent Application Publication
(10) Pub. No.: US 2024/0177867 A1
(43) Pub. Date: May 30, 2024

(54) METHOD, DEVICE, COMPUTER READABLE RECORDING MEDIUM, AND COMPUTER PROGRAM FOR PROVIDING MEDICAL INFORMATION

(71) Applicant: D&Life, Songpa-gu, Seoul (KR)

(72) Inventors: Tae-Won KIM, Seongnam-si (KR); Yu Sub SUNG, Seoul (KR); Sun Young KIM, Seoul (KR); Shin Kyo YOON, Seoul (KR); Seyoung SEO, Seoul (KR)

(73) Assignee: D&Life, Songpa-gu, Seoul (KR)

(21) Appl. No.: 18/555,808

(22) PCT Filed: Apr. 5, 2022
(86) PCT No.: PCT/KR2022/004825

§ 371 Date: Oct. 17, 2023

(30) Foreign Application Priority Data
Apr. 19, 2021 (KR) 10-2021-0050601

(51) Int. Cl. G16H 50/30; G16H 10/20; G16H 10/65

(52) U.S. Cl. CPC G16H 50/30; G16H 10/20; G16H 10/65

(57) ABSTRACT

The present disclosure relates to a method for providing a medical information using a device. a patient's status information is received. a medical information is provided for on a disease treatment step for each time domain over time based on the patient's status information. the medical information varies in detail on the disease treatment step for each time domain based on a number of items included in the patient's status information.

### BACKGROUND

### Technical Field

The present disclosure relates to a method, device, non-transitory computer readable recording medium, and computer program for providing medical information

### Related Art

Generally, patients receive explanations from medical professionals about the subsequent treatment steps for their illness after receiving medical care at the hospital. However, in many cases, these explanations from medical staff alone may not provide a clear understanding of the specific treatment processes.

For example, cancer patients often lack precise knowledge of their exact cancer stage, and most cancer patients are unable to plan long-term or short-term strategies because they are not well-informed about how the future treatment steps will unfold following their cancer diagnosis.

There are systems available where individuals can ask questions and receive real-time answers from medical professionals on websites and through the medical staff, but the websites providing cancer information are not regularly updated, and systems for asking questions and receiving real-time responses from medical professionals are limited to providing answers to frequently asked questions related to hospital operating hours, medical services offered, and similar topics.

Therefore, after receiving a diagnosis, patients find themselves in a situation where they lack detailed information about the specific future treatment steps.

### SUMMARY OF INVENTION

An objective of the present disclosure is to provide a method, device, non-transitory computer-readable recording medium, and computer program for providing medical information.

In addition, another objective of the present disclosure is to provide a patient's detailed medical information regarding a disease treatment step for a time domain over time by using the aforementioned method, device, computer-readable recording medium, and computer program for providing medical information.

However, the objectives of the present disclosure are not limited to those mentioned above, and other objectives not mentioned above may be clearly understood by those skilled in the art to which the present disclosure pertains from the description below.

In accordance with one aspect of the present disclosure, there is provided a method for providing a medical information using a medical information providing device, the method comprising: receiving a patient's status information: and providing medical information on a disease treatment step for each time domain over time based on the patient's status information, wherein the medical information varies in detail on the disease treatment step for each time domain based on a number of items included in the patient's status information.

Preferably, the disease treatment step for each time domain includes a disease treatment step for a time domain at a time when the patient's status information is received, a disease treatment step for a time domain before the point when the patient's status information is received, and a disease treatment step for a time domain after the point when the patient's status information is received.

Preferably, the disease treatment step for the time domain after the point when the patient's status information is received is provided at the point and includes at least two selectable disease treatment methods, and a number of disease treatment methods in the time domain after the point when the patient's status information is received becomes less as the patient's status information includes more items.

Preferably, the receiving of the patient's status information includes extracting the patient's status information from a card or QR code including the patient's medical information provided by medical staff.

Preferably, the patient's status information includes at least one of the patient's personal

information, disease information, treatment information, and genetic information.

Preferably, the patient's personal information contains at least one of the patient's name, gender, and date of birth, and the patient's disease information contains at least one of the patient's diagnosis name, the patient's disease stage, and a location of the patient's metastasis.

Preferably, the patient's treatment information contains at least one of the patient's surgery eligibility, the patient's surgery date, the patient's disease treatment date, and the patient's treatment medication.

Preferably, the medical information is information on the patient's cancer treatment step and includes at least one of disease information, treatment information, dietary information, rehabilitation information, similar case information, and clinical trial information which are related to the patient's disease stage.

Preferably, the receiving of the patient's status information includes: providing a user with at least one pre-stored question: receiving the user's response to the provided question as the patient's status information; and selecting an additional question to be provided based on the user's response and providing the selected question to the user.

In accordance with another aspect of the present disclosure, there is provided a non-transitory computer-readable recording medium having a computer program stored therein, wherein the computer program stores instructions which, when executed by a processor, cause the processor to perform operations comprising: receiving a patient's status information; and providing medical information on a disease treatment step for each time domain over time based on the patient's status information, wherein the medical information varies in detail on the disease treatment step for each time domain based on a number of items included in the patient's status information.

In accordance with a still another aspect of the present disclosure, there is provided a medical information providing device comprising: an input/output unit configured to receive a patient's status information: a memory: and a processor electrically connected to the memory, wherein the processor is configured to provide medical information on the patient's disease treatment step for each time domain over time based on the patient's status information, wherein the medical information varies in detail on the disease treatment step for each time domain based on a number of items included in the patient's status information.

By inputting a patient's status information through the medical information providing device 100 according to one aspect of the present disclosure, the user may receive real-time medical information based on the patient's status information, for each disease (e.g., cancer) treatment step within each time domain over time, the medical information which is information on illness, diagnosis, treatment, post-treatment management, palliative/hospice care, clinical trials, side effect management, rehabilitation/exercise, dietary guidance, and similar cases related to the patient's disease.

In addition, since the user is provided with customized information for each patient, it is possible to create a stable treatment plan and understand in detail the information needed to fight a disease based on the customized information for each patient.

However, effects of the present disclosure are not limited to the effects mentioned above, and other effects not mentioned will be clearly understood by those skilled in the art from the description below.

### BRIEF DESCRIPTION OF THE DRAWINGS

FIG. 1 is a block diagram of a medical information providing device according to an aspect of the present disclosure.

FIGS. 2 to 5 are diagrams to explain examples of medical information provided by a medical information providing device according to an aspect of the present disclosure.

FIG. 6 is an exemplary flowchart of a procedure for a medical information providing method according to an aspect of the present disclosure.

### DESCRIPTION OF EXEMPLARY EMBODIMENTS

The advantages and features of embodiments and methods of accomplishing these will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

In describing the embodiments of the present disclosure, if it is determined that detailed description of related known components or functions unnecessarily obscures the gist of the present disclosure, the detailed description thereof will be omitted. Further, the terminologies to be described below are defined in consideration of functions of the embodiments of the present disclosure and may vary depending on a user's or an operator's intention or practice. Accordingly, the definition thereof may be made on a basis of the content throughout the specification.

Hereinafter, embodiments of the present disclosure will be described in detail with reference to the attached drawings.

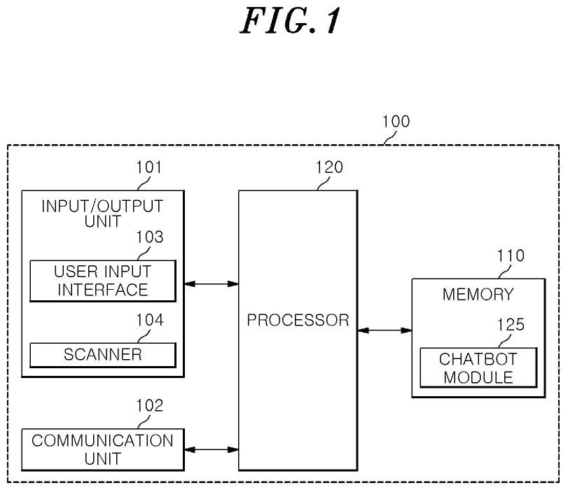

FIG. 1 is a block diagram of a medical information providing device according to an aspect of the present disclosure.

Referring to FIG. 1, a medical information providing device 100 according to an aspect of the present disclosure may include an input/output unit 101, a communication unit 102, a memory 110, and/or a processor 120, but not limited thereto, and may further include a chatbot module 125.

For example, the input/output unit 101 may transmit commands or data input from a user or another external device to other component(s) of the medical information providing device 100 according to one aspect, or may output commands or data received from other component(s) of the medical information providing device 100 to the user or an external device.

For example, the input/output unit 101 may receive a patient's status information.

Here, the patient's status information may include at least one of the patient's personal information, disease information, and treatment information, but not limited thereto, and may further include the patient's genetic information.

The patient's personal information may include at least one of the patient's name, gender, and date of birth (or birthdate).

The patient's disease information may be information on the patient's disease and may include at least one of the following: the patient's diagnosis, the patient's disease stage, and a location of the patient's metastasis (e.g., metastatic cancer).

The patient's treatment information is related to the patient's treatment timing and may include at least one of the following: the patient's surgery eligibility, the patient's surgery date, the patient's disease treatment date (e.g., radiation therapy date or start date of chemotherapy), and the patient's treatment medication (e.g., chemotherapy drugs).

The patient's genetic information may include at least one of the patient's next generation sequencing (NGS) result, immunostaining result, polymerase chain reaction (PCR) result, and fluorescence in situ hybridization (FISH) result.

In one embodiment, the input/output unit 101 may include a user input interface 103 and may receive the patient's status information input from a user through the user input interface 103. Here, the user input interface 103 may include a keyboard, a mouse, a touch pad, etc.

Here, the user may be a patient or a guardian, but not limited thereto.

In another embodiment, the input/output unit 101 may include a scanner 104 capable of scanning a barcode or QR code, and may receive the patient's status information through a card or QR code provided by the medical staff by scanning the card or QR code with the scanner 104, which includes the patient's medical information.

Here, the patient's medical information provided by the medical staff relates to detailed

status information regarding the patient's current disease and may include, but is not limited to, information on the patient's current treatment step and the patient's medical record.

At this point, when the user inputs the patient's status information through a card or QR code including the patient's medical information provided by the medical staff, rather than directly through the user interface 103, more detailed patient status information may be input.

Here, the card may be a physical card or a mobile card stored on the user's terminal, and the QR code may be a QR code printed on a QR code card, a paper-based QR code, or a QR code stored on the user's terminal (e.g., a QR code stored in an application).

For example, the card or QR code may include not only the patient's medical information provided by the medical staff but also the patient's status information, and the patient's medical information included in the card or QR code, provided by the medical staff, may be continuously updated by the medical staff and provided to the patient.

Meanwhile, the medical information providing device 100 according to one aspect of the present disclosure may include a chatbot module 125, and more specifically, when executed by the processor 120, the chatbot module 125 may provide at least one of pre-stored questions to the user, receive the user's response to the provided question as patient's status information, select an additional question to provide to the user based on the user's response, and transmit the input patient's status information to the input/output unit 101.

Therefore, the input/output unit 101 may receive a patient's status information based on a chatbot.

The communication unit 102 may support establishment of a wired or wireless communication channel between the medical information providing device 100 and an external device and communication through the established communication channel.

The memory 110 may store various data used by at least one component of the medical information providing device 100 (the processor 120, the input/output unit 101, and/or the communication unit 102), for example, input data or output data related to software (e.g., programs) and associated commands. The memory 110 may include a volatile memory or a non-volatile memory.

The processor 120 (also referred to as a control unit, controller, or control circuit) may control at least one of other components of the medical information providing device 100 (e.g., hardware components (e.g., the input/output unit 101, the communication unit 102, and/or the memory 110), or software components, and perform various data processing and operations.

In addition, the processor 120 may load received commands or data from at least one of the other components into a volatile memory for processing and store various data in a non-volatile memory.

To this end, the processor 120 may be implemented as a dedicated processor (e.g., an embedded processor) dedicated to perform corresponding operations, or implemented as a general-purpose processor (e.g., a CPU, an application processor, or a Microcontroller Unit (MCU)) capable of performing the corresponding operations by executing one or more software programs stored in a memory device.

More specifically, based on the patient's status information received through the input/output unit 101, the processor 120 may provide medical information regarding a disease treatment step for each time domain over time.

In other words, based on the patient's status information received through the input/output unit 101, the processor 120 may provide medical information regarding the entire treatment journey (e.g., pre-treatment, treatment, and post-treatment) of the patient's disease (e.g., “cancer”).

In this case, medical information provided through the processor 120 may vary in detail regarding a disease treatment step for each time domain depending on the number of items included in the patient's status information.

Here, the disease treatment step for each time domain over time may include a disease treatment step for a time domain at a point of receiving the patient's status information, a disease treatment step for a time domain before the point when the patient's status information is received, and a disease treatment step for a time domain after the point when the patient's status information is received.

For example, the time domain at the point when the patient's status information is received may correspond to a disease treatment step for a time domain of “during treatment”, the time domain before the point when the patient's status information is received may correspond to a disease treatment step for a time domain of “pre-treatment”, and the time domain after the point when the patient's status information is received may correspond to a disease treatment step for a time domain of “post-treatment”.

Meanwhile, regarding the disease treatment step for the time domain after the point when the patient's status information is received, the processor 120 may provide medical information regarding the disease treatment step for the time domain after the point when the patient's status information is received.

Furthermore, the disease treatment step for the time domain after the point when the patient's status information is received may include at least two optional disease treatment methods, and when there are more items included in the patient's status information, there are fewer available disease treatment methods of the disease treatment step for the time domain after the point when the patient's status information is received.

For example, when there is a greater number of items included in the patient's status information, the processor 120 may provide information with fewer optional disease treatment methods of the disease treatment step for the time domain after the point when the patient's status information is received, and when there is a fewer number of items included in the patient's status information the processor 120 may provide information with more optional disease treatment methods of the disease treatment step for the time domain after the point when the patient's status information is received.

This is because when the patient's status information is few, there may be a greater number of optional disease treatment methods available in the future, whereas when the patient's status information is abundant, there may be a fewer number of optional disease treatment methods based on the patient's status information.

Meanwhile, based on the patient's status information, the processor 120 may provide the patient's medical information regarding a disease treatment step for each time domain over time and then additionally provide medical information regarding a disease treatment step for one of the following time domains: a time domain at a point of receiving the patient's status information based on a user's input, a time domain before the point when the patient's status information is received, or a time domain after the point when the patient's status information is received.

For example, when providing medical information regarding a disease treatment step for the time domain of “during treatment” which corresponds to the time domain at the point when the patient's status information is received, the processor 120 may provide medical information regarding a disease treatment method included in “during treatment” which corresponds to the time domain at the point when the patient's status information is received based on a user's input, medical information regarding a disease treatment method included in the disease treatment step for the time domain of “pre-treatment” which corresponds to the time domain before the point when the patient's status information is received, or medical information regarding a disease treatment method included in the disease treatment step for the time domain of “post-treatment” which corresponds to the time domain after the point when the patient's status information is received.

Medical information provided through the processor 120 may be information on the patient's cancer treatment step and may include at least one of the following: disease information (or illness information) related to the patient's disease stage, treatment information, dietary information, rehabilitation information, similar case information related to the patient's disease, and clinical trial information.

In more detail, the medical information provided through the processor 120 may include, for each disease (e.g., cancer) treatment step within each time domain, information on illness, diagnosis, treatment, post-treatment management, palliative/hospice care, clinical trials, side effects management, rehabilitation/exercise, dietary information, and similar cases related to the patient's disease.

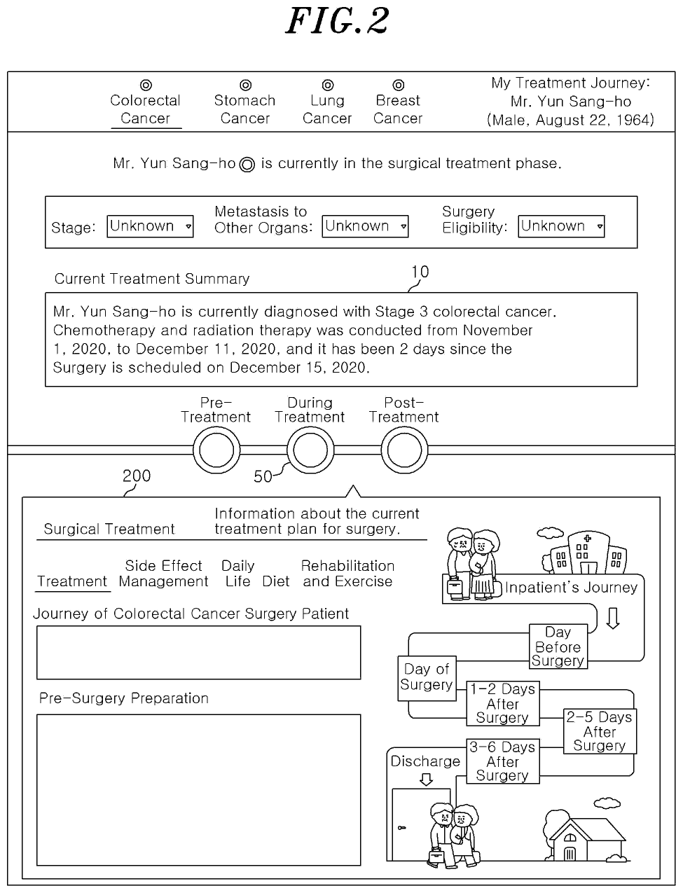

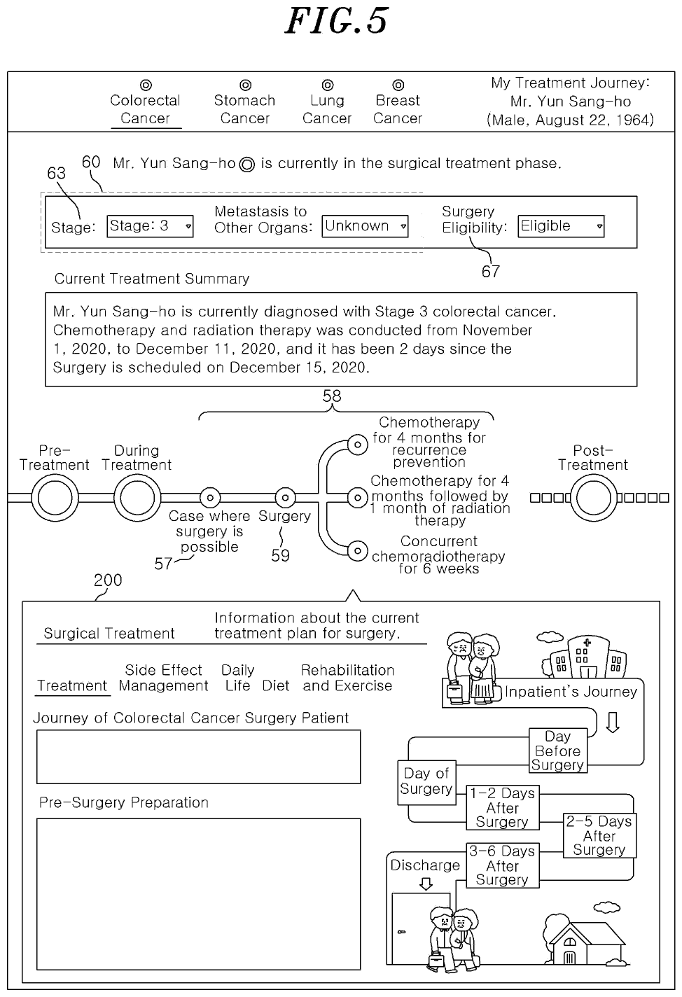

Hereinafter, examples of medical information provided by the medical information providing device 100 according to one aspect of the present disclosure will be described in detail through FIGS. 2 to 5.

FIGS. 2 to 5 are diagrams to explain examples of medical information provided by a medical information providing device according to an aspect of the present disclosure.

First, referring to FIG. 2, when the input/output unit 101 receives a patient's personal information among the patient's status information, the processor 120 may select provide medical information 200 regarding a disease treatment step 50 for a time domain of “during treatment” which corresponds to a time domain at a point when the patient's status information is received.

In addition, when the patient's status information is received, the processor 120 may provide summary information 10 of the patient's current treatment status.

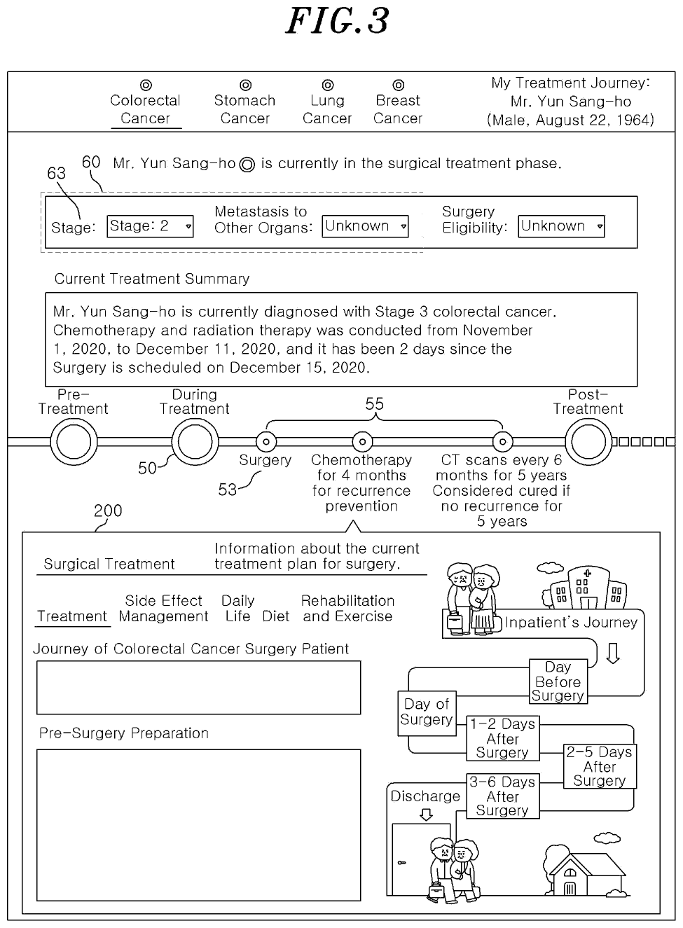

Referring to FIG. 3, when the patient's disease stage information 63 (Disease Stage, 2) is received for the patient's disease information 60 among the patient's status information, the input/output unit 101 may provide medical information 200 regarding a step 53 (Surgery) corresponding to the received patient's disease information, from among medical methods 55

(Surgery, Chemotherapy for 4 months for recurrence prevention, and CT scans every 6 months for 5 years, considered cured if no recurrence for 5 years) included in a disease treatment step 50 for the time domain at the point when the patient's status information is received.

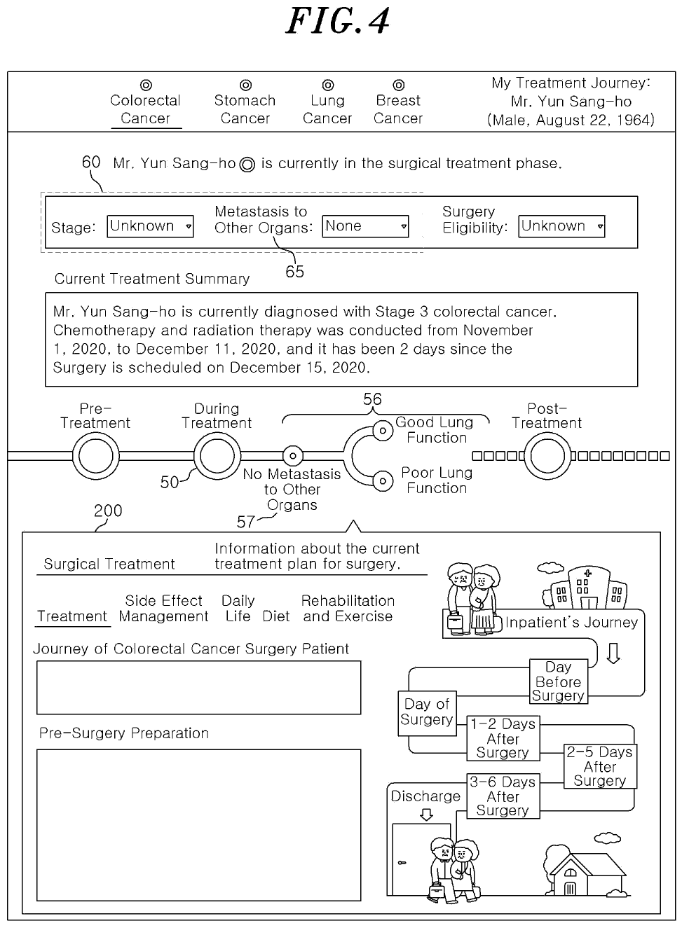

Referring to FIG. 4, when the patient's metastasis information 65 (no metastasis to other organs) is received for the patient's disease information 60 among the patient's status information, the input/output unit 101 may provide medical information 200 regarding a step 57 (no metastasis to other organs) corresponding to the received patient's disease information, from among treatment methods 56 (no metastasis to other organs, good lung function, and poor lung function) included in a disease treatment step 50 for a time domain of “during treatment” which corresponds to a time domain at a point when the patient's status information is received.

In this case, there may be two available disease treatment methods (good lung function and poor lung function) in a disease treatment step for a time domain after the point when the patient's status information is received.

When the patient's disease stage information 63 (Disease Stage, 3) is received for the patient's disease information among the patient's status information and the patient's surgery eligibility information 67 (Eligible) is received for the patient's treatment information, the input/output unit 101 may provide medical information 200 regarding a step 59 (Surgery) corresponding to the received patient's disease information and treatment information, from among treatment methods 58 (Case where surgery is possible, Surgery, Chemotherapy for 4 months for recurrence prevention, Chemotherapy for 4 months followed by 1 month of radiation therapy, and Concurrent chemoradiotherapy for 6 weeks) included in a disease treatment step 50 for a time domain of “during treatment” which corresponds to a time domain at a point when the patient's status information is received. In this case, there may be three available disease treatment methods (Chemotherapy for 4 months for recurrence prevention, Chemotherapy for 4 months followed by 1 month of radiation therapy, and Concurrent chemoradiotherapy for 6 weeks) in a disease treatment step for a time domain after the point when the patient's status information is received.

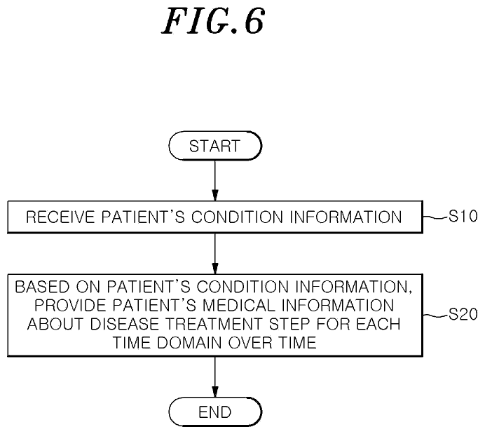

FIG. 6 is an exemplary flowchart of a procedure for a medical information providing method according to an aspect of the present disclosure. A medical information providing method of FIG. 6 may be performed by the medical information providing device 100 shown in FIG. 1. In addition, the medical information providing method shown in FIG. 6 is merely illustrative.

Referring to FIG. 6, the input/output unit 101 may receive a patient's status information (S10).

In one embodiment, the input/output unit 101 may receive the patient's status information input from a user through the user interface 103.

In another embodiment, the input/output unit 101 may receive the patient's status information through a card or QR code including the patient's medical information provided by medical staff. Specifically, the input/output unit 101 may recognize the card or QR code and obtain the patient's status information included in the card or QR code.

In yet another embodiment, when the processor 120 of the medical information providing device 100 according to one aspect of the present disclosure provides the user with at least one question pre-stored in the memory 110 through the input/output unit 101, the input/output unit 101 may receive the user's response to the provided question as the patient's status information.

Thereafter, based on the patient's status information received in the input/output unit 101, the processor 120 may provide medical information regarding the patient's disease treatment step for each time domain over time (S20).

Here, the medical information provided by the processor 120 is information about the patient's cancer treatment step, and may include at least one of disease information, treatment information, dietary information, rehabilitation information, similar case information, and clinical trial information which are related to the patient's disease stage.

In more detail, the medical information provided through the processor 120 may include, for each disease (e.g., cancer) treatment step within each time domain, information on illness, diagnosis, treatment, post-treatment management, palliative/hospice care, clinical trials, side effects management, rehabilitation/exercise, dietary information, and similar cases related to the patient's disease.

As discussed above, by inputting a patient's status information through the medical information providing device 100 according to one aspect of the present disclosure, the user may receive real-time medical information based on the patient's status information, for each disease (e.g., cancer) treatment step within each time domain over time, the medical information which is information on illness, diagnosis, treatment, post-treatment management, palliative/hospice care, clinical trials, side effect management, rehabilitation/exercise, dietary guidance, and similar cases related to the patient's disease.

Therefore, by entering the patient's status information through the medical information providing device 100 according to one aspect of the present disclosure, the user who is not a medical expert, such as a patient or guardian, may be provided with accurate and detailed information on the patient.

In addition, since the user is provided with customized information for each patient, it is possible to create a stable treatment plan and understand in detail the information needed to fight a disease based on the customized information for each patient.

Combinations of respective blocks of block diagrams attached herein and respective steps of a sequence diagram attached herein may be carried out by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the steps of the flowchart. These computer program instructions may also be stored in a computer usable or computer readable memory that can direct a computer or other programmable data processing apparatuses to function in a particular manner, such that the instructions stored in the computer usable or computer readable medium can produce an article of manufacture including instructions that implement the function specified in the blocks of the flowcharts. The computer program instructions may also be loaded onto a computer or other programmable data processing apparatuses to cause a series of operational steps to be performed on the computer or other programmable apparatuses to produce a computer implemented process such that the instructions that are executed on the computer or other programmable apparatuses provide processes for implementing the functions specified in the blocks of the flowcharts.

In addition, each block in the flowchart may represent a module, segment, or portion of code, which comprises one or more executable instructions for implementing the specified logical function(s). Furthermore, it should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

What is claimed is:

1. A medical information providing method performed by a medical information providing device, the method comprising:
receiving a patient's status information; and
providing medical information on a disease treatment step for each time domain over time based on the patient's status information,
wherein the medical information varies in detail on the disease treatment step for each time domain based on a number of items included in the patient's status information.

2. The medical information providing method of claim 1,
wherein the disease treatment step for each time domain includes: a disease treatment step for a time domain at a time when the patient's status information is received, a disease treatment step for a time domain before the point when the patient's status information is received, and a disease treatment step for a time domain after the point when the patient's status information is received.

3. The medical information providing method of claim 2, wherein:
the disease treatment step for the time domain after the point when the patient's status information is received is provided at the point and comprises at least two selectable disease treatment methods, and
a number of disease treatment methods in the time domain after the point when the patient's status information is received becomes less as the patient's status information includes more items.

4. The medical information providing method of claim 1, wherein the receiving of the patient's status information includes extracting the patient's status information from a card or QR code including the patient's medical information provided by medical staff.

5. The medical information providing method of claim 1, wherein the patient's status information includes at least one of the patient's personal information, disease information, treatment information, and genetic information.

6. The medical information providing method of claim 5, wherein:
the patient's personal information contains at least one of the patient's name, gender, and date of birth, and
the patient's disease information contains at least one of the patient's diagnosis name, the patient's disease stage, and a location of the patient's metastasis.

7. The medical information providing method of claim 5, wherein the patient's treatment information contains at least one of the patient's surgery eligibility, the patient's surgery date, the patient's disease treatment date, and the patient's treatment medication.

8. The medical information providing method of claim 1, wherein the medical information is information on the patient's cancer treatment step and includes at least one of disease information, treatment information, dietary information, rehabilitation information, similar case information, and clinical trial information which are related to the patient's disease stage.

9. The medical information providing method of claim 1, wherein the receiving of the patient's status information includes:
providing a user with at least one pre-stored question:
receiving the user's response to the provided question as the patient's status information; and
selecting an additional question to be provided based on the user's response and providing the selected question to the user.

10. A medical information providing device comprising:
an input/output unit configured to receive a patient's status information:
a memory; and
a processor electrically connected to the memory,
wherein the processor is configured to provide medical information on the patient's disease treatment step for each time domain over time based on the patient's status information,
wherein the medical information varies in detail on the disease treatment step for each time domain based on a number of items included in the patient's status information.

11. The medical information providing device of claim 10, wherein the disease treatment step for each time domain includes: a disease treatment step for a time domain at a point when the patient's status information is received, a disease treatment step for a time domain before the point when the patient's status information is received, and a disease treatment step for a time domain after the point when the patient's status information is received.

12. The medical information providing device of claim 11, wherein:
the disease treatment step for the time domain after the point when the patient's status information is received is provided at the point and comprises at least two selectable disease treatment methods, and
a number of disease treatment methods in the time domain after the point when the patient's status information is received becomes less as the patient's status information includes more items.

13. The medical information providing device of claim 10, wherein the input/output unit is further configured to receive the patient's status information extracted from a card or QR code including the patient's medical information provided by medical staff.

14. The medical information providing device of claim 10, wherein the patient's status information includes at least one of the patient's personal information, disease information, treatment information, and genetic information.

15. The medical information providing device of claim 14, wherein:
the patient's personal information contains at least one of the patient's name, gender, and date of birth, and
the patient's disease information contains at least one of the patient's diagnosis name, the patient's disease stage, and a location of the patient's metastasis.

16. The medical information providing device of claim 14, wherein the patient's treatment information contains at least one of the patient's surgery eligibility, the patient's surgery date, the patient's disease treatment date, and the patient's treatment medication.

17. The medical information providing device of claim 10, wherein the medical information is information on the patient's cancer treatment step and includes at least one of disease information, treatment information, dietary information, rehabilitation information, similar case information, and clinical trial information which are related to the patient's disease stage.

18. The medical information providing device of claim 10, further including:
a chatbot module that is, when executed by the processor, configured to provide a user with at least one pre-stored question, receive the user's response to the provided question the patient's status information, select an additional question to be provided to the user based on the user's response, provide the selected question to the user, and transmit the input patient status information to the input/output unit.

19. A non-transitory computer-readable recording medium having a computer program stored therein, wherein the computer program stores instructions which, when executed by a processor, cause the processor to perform operations comprising:
receiving a patient's status information; and
providing medical information on a disease treatment step for each time domain over time based on the patient's status information,
wherein the medical information varies in detail on the disease treatment step for each time domain based on a number of items included in the patient's status information.
